(19) United States
(12) Patent Application Publication
(10) Pub. No.: US 2023/0355483 A1
(43) Pub. Date: Nov. 9, 2023

(54) TWO-PHASE COMPOSITION COMPRISING AT LEAST ONE HYDROCARBON OIL AND AT LEAST ONE ESTER OF CITRIC ACID, WHEREIN THE COMPOSITION IS PREFERABLY A CLEANSING COMPOSITION

(71) Applicant: L'OREAL, PARIS (FR)

(72) Inventors: Natsuno MISHINA, Kawasaki-shi (JP); Masanori ORITA, Kawasaki-shi (JP); Sho WATANABE, Kawasaki-shi (JP)

(21) Appl. No.: 18/246,033

(22) PCT Filed: Sep. 17, 2021
(86) PCT No.: PCT/JP2021/035219

§ 371 Date: Mar. 20, 2023

(30) Foreign Application Priority Data
Sep. 28, 2020 (JP) 2020-162248
Oct. 30, 2020 (FR) 2011131

(51) Int. Cl. A61K 8/03; A61K 8/92; A61K 8/37; A61K 8/35; A61K 8/39; A61Q 1/14

(52) U.S. Cl. CPC A61K 8/03; A61K 8/92; A61K 8/37; A61K 8/35; A61K 8/39; A61Q 1/14; A61K 2800/882

(57) ABSTRACT

The present invention relates to a two-phase composition comprising an oil phase comprising: an oil phase comprising (a) at least one hydrocarbon oil; and an aqueous phase comprising (b) at least one ester of citric acid, and (c) water, wherein the amount of the (a) hydrocarbon oil in the composition is 6% by weight or more, preferably 7% by weight or more, and more preferably 8% by weight or more, relative to the total weight of the composition. The composition according to the present invention has two visually distinct phases when not being mixed. However, the composition according to the present invention can be transformed into a single phase composition when being mixed. The single phase composition, such as an emulsion, can provide good cosmetic effects such as good make-up removability, without discomfort.

### TECHNICAL FIELD

The present invention relates to a two-phase or bi-phase composition which has two visually distinct phases and is capable of transforming into a single phase composition that can transform again into the two-phase or bi-phase composition.

### BACKGROUND ART

Compositions having two visually distinct phases, especially an aqueous phase and an oily phase, which are not emulsified in each other at rest, are generally referred to as “two-phase” (or “bi-phase”) compositions. They differ from emulsions by the fact that, at rest, the two phases are separate and form a single interface between the two phases, instead of forming multiple interfaces between a continuous phase and each of the discontinuous phases which are present in emulsions.

Such two-phase compositions have already been described, for example, in EP-A-370 856 and EP-A-603 080, especially for removing make-up from around the eyes.

When using, a two-phase composition requires mixing, such as shaking, in order to form a single phase composition, such as a “temporary” emulsion, which is then applied onto a keratin substance such as skin. This single phase composition needs to provide target cosmetic effects such as good make-up removability. It is preferable if the single phase composition can provide cosmetic effects without providing discomfort such as stickiness.

Thus, there is a need for a two-phase or bi-phase composition with two visually distinct phases which can be transformed into a single phase composition when being mixed, wherein the single phase composition can provide good cosmetic effects such as good make-up removability, without discomfort, and can return to its initial state, i.e., a two-phase or bi-phase composition.

### DISCLOSURE OF INVENTION

An objective of the present invention is to provide a two-phase composition which can be transformed into a single phase composition when being mixed, wherein the single phase composition can provide good cosmetic effects such as good make-up removability, without discomfort, and can return to a two-phase composition.

The above objective can be achieved by a two-phase composition comprising:
an oil phase comprising (a) at least one hydrocarbon oil; and
an aqueous phase comprising (b) at least one ester of citric acid, and (c) water,
wherein
the amount of the (a) hydrocarbon oil in the composition is 6% by weight or more, preferably 7% by weight or more, and more preferably 8% by weight or more, relative to the total weight 50 of the composition.

The composition according to the present can transform into a single phase composition. The single phase composition can transform into a two phase composition.

The (a) hydrocarbon oil may be a volatile hydrocarbon oil, preferably a volatile branched C₈-C₁₆ hydrocarbon oil, and more preferably selected from the group consisting of isohexadecane, isodecane, isododecane, and a mixture thereof.

The amount of the (a) hydrocarbon oil(s) in the composition according to the present invention may be from 6% to 50% by weight, preferably from 7% to 45% by weight, and more preferably from 8% to 40% by weight, relative to the total weight of the composition.

The (b) ester of citric acid may be represented by the following chemical formula (I):

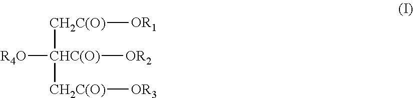

wherein
R₁, R₂ and R₃ independently represent a hydrogen atom, a saturated or unsaturated, linear or branched C₁-C₃₀ hydrocarbon group, or a saturated or unsaturated cyclic C₃-C₃₀ alkyl hydrocarbon, wherein at least one of R₁, R₂ and R₃ is not a hydrogen atom, and
R₄ represents a hydrogen atom or an R′₄—CO— group in which R′₄ represents a saturated or unsaturated, linear or branched C₁-C₈ hydrocarbon group, or a saturated or unsaturated cyclic C₃-C₈ hydrocarbon group.

The (b) ester of citric acid may be selected from the group consisting of triethyl citrate, tributyl citrate, trioctyl citrate, triethyl acetylcitrate, tribuyl acetylcitrate, tri(2-ethylhexyl) acetylcitrate, and a mixture thereof.

The amount of the (b) ester(s) of citric acid in the composition according to the present invention may be from 0.1% to 10% by weight, preferably from 0.5% to 5% by weight, and more preferably from 1% to 3% by weight, relative to the total weight of the composition.

The amount of the (c) water in the composition according to the present invention may be from 30% to 93% by weight, and preferably from 35% to 85% by weight, relative to the total weight of the composition.

The composition according to the present invention may further comprise at least one non-volatile oil which is not the (a) hydrocarbon oil, preferably at least one non-volatile non-polar oil, and more preferably non-volatile ether oil.

The composition according to the present invention may further comprise polar oil such as ester oil in an amount of from 0.01% to 20% by weight, and preferably from 0.1% to 10% by weight, relative to the total weight of the composition.

The amount of the oil phase in the composition according to the present invention may be from 7% to 70% by weight, and preferably from 15% to 65% by weight, relative to the total weight of the composition.

The amount of the aqueous phase in the composition according to the present invention may be from 30% to 93% by weight, and preferably from 35% to 85% by weight, relative to the total weight of the composition.

The composition according to the present invention may be a cosmetic composition, preferably a cleansing cosmetic composition, and more preferably a cleansing cosmetic composition for skin and eyelashes.

The present invention also relates to a cosmetic process for a keratin substance, comprising: mixing the composition according to the present invention to form a single phase composition; and applying the single phase composition onto the keratin substance.

### BEST MODE FOR CARRYING OUT THE INVENTION

After diligent research, the inventors have discovered that it is possible to provide a two-phase composition which can be transformed into a single phase composition when being mixed, wherein the single phase composition can provide good cosmetic effects such as good make-up removability, without discomfort, and can return to a two-phase composition.

Thus, one of the aspects of the present invention relates to a two-phase composition comprising:
an oil phase comprising (a) at least one hydrocarbon oil; and
an aqueous phase comprising (b) at least one ester of citric acid, and (c) water,
wherein
the amount of the (a) hydrocarbon oil in the composition is 6% by weight or more, preferably 7% by weight or more, and more preferably 8% by weight or more, relative to the total weight of the composition.

The composition according to the present invention has two visually distinct phases when not being mixed. However, the composition according to the present invention can be transformed into a single phase composition by mixing when being used. The single phase composition, such as an emulsion, can provide good cosmetic effects such as good make-up removability, without discomfort such as stickiness. The single phase composition can return to a two-phase composition when not being used.

The composition according to the present invention is stable such that it can maintain an oil phase and an aqueous phase separately when not being mixed. Therefore, the two-phase or bi-phase aspect of the composition according to the present invention can be maintained during storage and the like of the composition according to the present invention.

In particular, the composition according to the present invention could be stable such that the aspect and/or smell of the composition can remain unchanged for a long period of time under various temperature and storage conditions. For example, the composition according to the present invention could be stable at a lower temperature, with or without shaking. Accordingly, the composition according to the present invention could be stored well, in particular at a lower temperature even during transportation by automobiles which may cause vibrations and the like.

When being used, the composition according to the present invention is mixed. The two phases in the composition according to the present invention are an oil phase comprising at least one oil and an aqueous phase comprising water. Therefore, when mixing the composition according to the present invention, one of the two phases can be dispersed in the other phase to form an O/W or a W/O composition, such as an O/W or a W/O emulsion, which is visually uniform or forms a single phase.

The mixing of the composition according to the present invention can be easily performed. For example, the mixing of the composition according to the present invention can be performed by shaking with the hands. After mixing the composition according to the present invention, the composition can form and maintain a single phase for a certain period of time without mixing again.

According to the present invention, the single phase composition formed by the two-phase composition can provide good cosmetic effects such as good make-up removability. Therefore, the composition according to the present invention can be used as a cosmetic composition, preferably a cleansing cosmetic composition, and more preferably a cleansing cosmetic composition for skin and eyelashes.

Further, the single phase composition formed by the two-phase composition according to the present invention can provide a good texture such as a smooth feeling to touch. In addition, it could provide less skin-scaling. The skin-scaling reflects the relatively large loss of the outer layer of the epidermis of skin, and can make the surface of the skin rough. Therefore, the composition according to the present invention can provide a smooth skin finish.

Furthermore, the single phase composition formed by the two-phase composition according to the present invention could be more comfortable, for example, it could be less irritating. Therefore, the composition according to the present invention could be, for example, mild on the skin, in particular sensitive skin such as the skin around the eyes.

If the two-phase composition according to the present invention before being mixed is transparent, the single phase composition formed by mixing the two-phase composition can also be transparent.

The single phase composition formed by the two-phase composition according to the present invention could rapidly return to the two-phase composition again, after a certain period of time, by causing phase separation to re-form two visually distinct phases again. According to the present invention, the interface between the two re-formed phases could be clean such that no precipitation is present at the interface.

The composition according to the present invention is useful as, in particular, a make-up remover, preferably a make-up remover for keratin fibers, and more preferably a make-up remover for eyelashes, i.e., a mascara.

Hereafter, the composition according to the present invention will be described in a detailed manner.

[Composition]

(Hydrocarbon Oil)

The composition according to the present invention comprises (a) at least one hydrocarbon oil. If two or more hydrocarbon oils are used, they may be the same or different.

The (a) hydrocarbon oil can form the oil phase of the composition according to the present invention.

The term “hydrocarbon” here means a compound formed by carbon and hydrogen atoms, and does not comprise any heteroatom such as oxygen and nitrogen atoms.

Here, “oil” means a fatty compound or substance which is in the form of a liquid or a paste (non-solid) at room temperature (25° C.) under atmospheric pressure (760 mmHg).

The (a) hydrocarbon oil may be selected from aliphatic hydrocarbon oils, alicyclic hydrocarbon oils and aromatic hydrocarbon oils, preferably aliphatic hydrocarbon oils, and more preferably aliphatic and saturated hydrocarbon oils.

The (a) hydrocarbon oil may be volatile or non-volatile. It is preferable that the (a) hydrocarbon oil be volatile. In other words, it is preferable that the (a) hydrocarbon oil be a volatile hydrocarbon oil.

For the purposes of the present invention, the term “volatile” oil means an oil that is capable of evaporating on contact with keratin materials in less than one hour, at room temperature (25° C.) and atmospheric pressure (760 mmHg). The volatile oil may be a volatile cosmetic oil, which is liquid at room temperature, especially having a non-zero vapour pressure, at room temperature and atmospheric pressure, in particular having a vapour pressure ranging from 0.13 Pa to 40 000 Pa (10-3 to 300 mmHg), preferably ranging from 1.3 Pa to 13 000 Pa (0.01 to 100 mmHg) and preferentially ranging from 1.3 Pa to 1300 Pa (0.1 to 10 mmHg).

According to one preferred embodiment, the volatile hydrocarbon oil has a flash point of greater than 65° C., and better still greater than 80° C.

The (a) hydrocarbon oil may be chosen from volatile hydrocarbon oils containing from 8 to 16 carbon atoms, preferably volatile branched C₈-C₁₆ hydrocarbon oils, and more preferably volatile branched C₈-C₁₆ alkanes (also known as isoparaffins). It may be even more preferable the (a) hydrocarbon oil be selected from the group consisting of isohexadecane, isodecane, isododecane (also called 2,2,4,4,6-pentamethylheptane), and a mixture thereof.

On the other hand, the (a) hydrocarbon oil may also be a linear volatile alkane containing 7 to 17 carbon atoms, in particular 9 to 15 carbon atoms, and more particularly 11 to 13 carbon atoms. Mention may be made especially of n-nonadecane, n-decane, n-undecane, n-dodecane, n-tridecane, n-tetradecane, n-pentadecane and n-hexadecane, and a mixture thereof.

In particular, the (a) hydrocarbon oil may be selected from the group consisting of isohexadecane, isodecane, isododecane, and a mixture thereof

The amount of the (a) hydrocarbon oil(s) in the composition according to the present invention is 6% by weight or more, preferably 7% by weight or more, and more preferably 8% by weight or more, relative to the total weight of the composition.

The amount of the (a) hydrocarbon oil(s) in the composition according to the present invention may be 50% by weight or less, preferably 45% by weight or less and more preferably 40% by weight or less, relative to the total weight of the composition, with the proviso that the amount of the (a) hydrocarbon oil(s) is not zero.

The amount of the (a) hydrocarbon oil(s) in the composition according to the present invention may be from 6% to 50% by weight, preferably from 7% to 45% by weight, and more preferably from 8% to 40% by weight, relative to the total weight of the composition.

(Ester of Citric Acid)

The composition according to the present invention comprises (b) at least one ester of citric acid. If two or more esters of citric acid are used, they may be the same or different.

The (b) ester of citric acid may be represented by the following chemical formula (I):

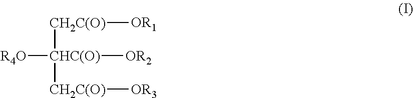

wherein
R₁, R₂ and R₃ independently represent a hydrogen atom, a saturated or unsaturated, linear or branched C₁-C₃₀ hydrocarbon group, or a saturated or unsaturated cyclic C₃-C₃₀ hydrocarbon group, wherein at least one of R₁, R₂ and R₃ is not a hydrogen atom, and
R₄ represents a hydrogen atom or an R′₄—CO— group in which R′₄ represents a saturated or unsaturated, linear or branched C₁-C₈ hydrocarbon group, or a saturated or unsaturated cyclic C₃-C₈ hydrocarbon group.

It is preferable that all of R₁, R₂ and R₃ be a saturated or unsaturated, linear or branched C₁-C₃₀ hydrocarbon group, or a saturated or unsaturated cyclic C₃-C₃₀ hydrocarbon group, more preferably a saturated or unsaturated, linear or branched C₁-C₃₀ hydrocarbon group, and even more preferably a linear or branched C₁-C₃₀ alkyl group.

It is preferable that R₄ be a hydrogen atom or an R′₄—CO— group in which R′₄ represents a saturated or unsaturated, linear or branched C₁-C₈ hydrocarbon group, more preferably a hydrogen atom or an R′₄—CO— group in which R′₄ represents a linear or branched C₁-C₈ alkyl group, and even more preferably a hydrogen atom or an R′₄—CO— group in which R′4 represents a methyl group.

It may be preferable that the (b) ester of citric acid be selected from the group consisting of triethyl citrate, tributyl citrate, trioctyl citrate, triethyl acetylcitrate, tribuyl acetylcitrate, tri(2-ethylhexyl) acetylcitrate, and a mixture thereof, more preferably the group consisting of triethyl citrate, tributyl citrate, triethyl acetylcitrate, tribuyl acetylcitrate, and a mixture thereof, and even more preferably ethyl citrate.

The amount of the (b) ester(s) of citric acid in the composition according to the present invention may be 0.1% by weight or more, preferably 0.5% by weight or more, and more preferably 1% by weight or more, relative to the total weight of the composition.

The amount of the (b) ester(s) of citric acid in the composition according to the present invention may be 10% by weight or less, preferably 5% by weight or less and more preferably 3% by weight or less, relative to the total weight of the composition, with the proviso that the amount of the (b) ester(s) of citric acid is not zero.

The amount of the (b) ester(s) of citric acid in the composition according to the present invention may be from 0.1% to 10% by weight, preferably from 0.5% to 5% by weight, and more preferably from 1% to 3% by weight, relative to the total weight of the composition.

(Water)

The composition according to the present invention comprises (c) water.

The (c) water can form an aqueous phase of the composition according to the present invention.

The amount of the (c) water in the composition according to the present invention may be 30% by weight or more, and preferably 35% by weight or more, relative to the total weight of the composition.

The amount of the (c) water in the composition according to the present invention may be 93% by weight or less, and preferably 85% by weight or less, relative to the total weight of the composition, with the proviso that the amount of the (c) water is not zero.

The amount of the (c) water in the composition according to the present invention may be from 30% to 93% by weight, and preferably from 35% to 85% by weight, relative to the total weight of the composition.

(Non-Volatile Oil)

The composition according to the present invention may comprise at least one non-volatile oil which is not the (a) hydrocarbon oil. If two or more non-volatile oils are used, they may be the same or different.

The non-volatile oil may be selected from silicone oils, non-silicone oils, and mixtures thereof.

Non-Volatile Silicone Oil:

The non-volatile silicone oil that may be used in the present invention may be chosen from silicone oils with a viscosity at 25° C. of greater than or equal to 8 centistokes (cSt) (8×10⁻⁶ m²/s) and less than 800 000 cSt, preferably between 10 and 600 000 cSt and preferably between 100 and 500 000 cSt. The viscosity of this silicone may be measured according to standard ASTM D-445.

Among these silicone oils, two types of oil may be distinguished, according to whether or not they contain phenyl. Thus, the non-volatile silicone oil may be selected from non-phenylated silicone oils and phenylated silicone oils.

Representative examples of these non-volatile non-phenylated silicone oils that may be mentioned include polydimethylsiloxanes (dimethicone); alkyl dimethicones; vinyl methyl methicones; and also silicones modified with optionally fluorinated aliphatic groups, or with functional groups such as hydroxyl, thiol and/or amine groups.

Thus, non-volatile non-phenylated silicone oils that may be mentioned include:
PDMSs comprising alkyl or alkoxy groups, which are pendent and/or at the end of the silicone chain, these groups each containing from 2 to 24 carbon atoms,
PDMSs comprising aliphatic groups, or functional groups such as hydroxyl, thiol and/or amine groups,
polyalkylmethylsiloxanes optionally substituted with a fluorinated group, such as polymethyltrifluoropropyldimethylsiloxanes,
polyalkylmethylsiloxanes substituted with functional groups such as hydroxyl, thiol and/or amine groups, such as dihydroxydimethylsiloxane (dimethiconol)
polysiloxanes modified with fatty acids, fatty alcohols or polyoxyalkylenes, and
mixtures thereof.

According to one embodiment, the non-volatile silicone oil is selected from non-volatile non-phenylated linear silicone oils.

The non-volatile non-phenylated linear silicone oil may be chosen especially from the silicones of formula:

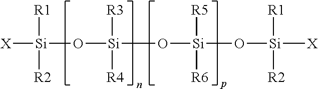

in which:
R₁, R₂, R₅ and R₆ are, together or separately, an alkyl radical containing 1 to 6 carbon atoms,
R₃ and R₄ are, together or separately, an alkyl radical containing from 1 to 6 carbon atoms, a vinyl radical, an amine radical or a hydroxyl radical,
X is an alkyl radical containing from 1 to 6 carbon atoms, a hydroxyl radical or an amine radical, and
n and p are integers chosen so as to have a fluid compound.

As non-volatile silicone oils that may be used according to the present invention, mention may be made of those for which:
the substituents R₁ to R₆ and X represent a methyl group, and p and n are such that the viscosity is 500 000 cSt, such as the product sold under the name SE30 by the company General Electric, the product sold under the name AK 500000 by the company Wacker, the product sold under the name Mirasil DM 500 000 by the company Bluestar, and the product sold under the name Dow Corning 200 Fluid 500 000 cSt by the company Dow Corning,
the substituents R₁ to R₆ and X represent a methyl group, and p and n are such that the viscosity is 300 000 cSt, such as the product sold under the name Wacker Belsil DM 300 000 by the company Wacker,
the substituents R₁ to R₆ and X represent a methyl group, and p and n are such that the viscosity is 60 000 cSt, such as the product sold under the name Dow Corning 200 Fluid 60000 CS by the company Dow Corning, and the product sold under the name Wacker Belsil DM 60 000 by the company Wacker,
the substituents R₁ to R₆ and X represent a methyl group, and p and n are such that the viscosity is 350 cSt, such as the product sold under the name Dow Corning 200 Fluid 350 CS by the company Dow Corning, and
the substituents R₁ to R₆ represent a methyl group, the group X represents a hydroxyl group, and n and p are such that the viscosity is 700 cSt, such as the product sold under the name Baysilone Fluid T0.7 by the company Momentive.

According to another embodiment, the non-volatile silicone oil is selected from non-volatile phenylated silicone oils.

Representative examples of these non-volatile phenylated silicone oils that may be mentioned include:
the phenyl silicone oils corresponding to the following formula

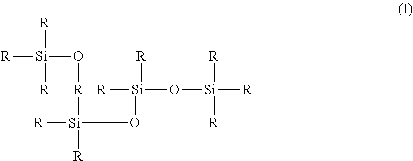

in which in formula: the groups represent, independently of each other, a methyl or a phenyl, with the proviso that at least one group R represents a phenyl. Preferably, in this formula, the phenyl silicone oil comprises at least three phenyl groups, for example at least four, at least five or at least six,
the phenyl silicone oils corresponding to the following formula;

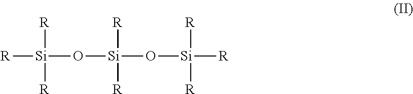

in which in formula (II): the groups R represent, independently of each other, a methyl or a phenyl, with the proviso that at least one group R represents a phenyl. Preferably, in this formula, the said organopolysiloxane comprises at least three phenyl groups, for example at least four or at least five. Mixtures of the phenyl organopolysiloxanes described previously may be used. Examples that may be mentioned include mixtures of triphenyl, tetraphenyl or pentaphenyl organopolysiloxanes,
the phenyl silicone oils corresponding to the following formula;

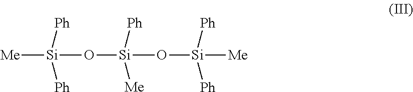 [SMILES: C[Si](O[Si](C)(c1ccccc1)c1ccccc1)(O[Si](C)(c1ccccc1)c1ccccc1)c1ccccc1]

in which in formula (III): Me represents methyl, Ph represents phenyl. Such a phenyl silicone is especially manufactured by Dow Corning under the reference PH-1555 HRI or Dow Corning 555 Cosmetic Fluid (chemical name: 1,3,5-trimethyl-1,1,3,5,5-pentaphenyl trisiloxane; INCI name: trimethyl pentaphenyl trisiloxane).
The reference Dow Corning 554 Cosmetic Fluid may also be used, the phenyl silicone oils corresponding to the following formula;

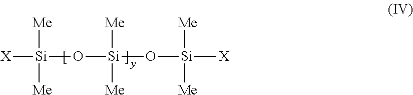

in which, in formula (IV), Me represents methyl, y is between 1 and 1000 and X represents —CH₂—CH(CH₃)(Ph).
the phenyl silicone oils corresponding to formula (V) below;

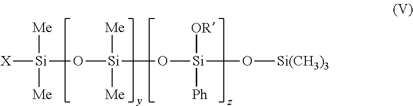

in which in formula (V): Me is methyl and Ph is phenyl, OR′ represents a group —OSiMe₃ and y is 0 or ranges between 1 and 1000, and z ranges between 1 and 1000, such that compound (V) is a non-volatile oil. According to a first embodiment, y ranges between 1 and 1000. Use may be made, for example, of trimethyl siloxyphenyl dimethicone, sold especially under the reference Belsil PDM 1000 sold by the company Wacker. According to a second embodiment, y is equal to 0. Use may be made, for example, of phenyl trimethylsiloxy trisiloxane, sold especially under the reference Dow Corning 556 Cosmetic Grade Fluid,
the phenyl silicone oils corresponding to formula (VI) below, and mixtures thereof;

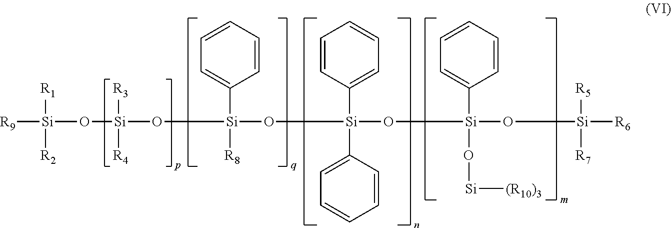

in which in formula (VI):
R₁ to R₁₀, independently of each other, are saturated or unsaturated, linear, cyclic or branched C₁-C₃₀ hydrocarbon-based radicals,
m, n, p and q are, independently of each other, integers between 0 and 900, with the proviso that the sum m+n+q is other than 0; preferably, the sum m+n+q is between 1 and 100. Preferably, the sum m+n+p+q is between 1 and 900 and better still between 1 and 800. Preferably, q is equal to 0,
the phenyl silicone oils corresponding to formula (VII) below, and mixtures thereof;

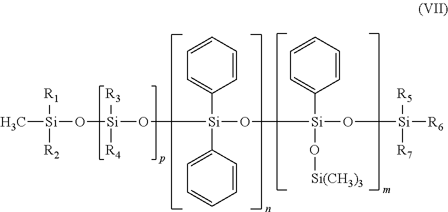

in which in formula (VII):
R₁ to R₆, independently of each other, are saturated or unsaturated, linear, cyclic or branched C₁-C₃₀ hydrocarbon-based radicals,
m, n and p are, independently of each other, integers between 0 and 100, with the proviso that the sum n+m is between 1 and 100; preferably, R₁ to R₆, independently of each other, represent a saturated, linear or branched C₁-C₃₀ and especially C₁-C₁₂ hydrocarbon-based radical and in particular a methyl, ethyl, propyl or butyl radical; R₁ to R₆ may especially be identical, and in addition may be a methyl radical; Preferably, m=1 or 2 or 3, and/or n=0 and/or p=0 or 1 may apply, in formula (VII),
the phenyl silicone oils corresponding to formula (VIII), and mixtures thereof:

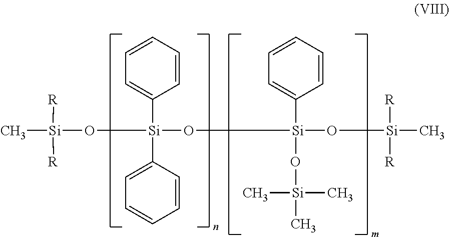

in which in formula (VIII):
R is a C₁-C₃₀ alkyl radical, an aryl radical or an aralkyl radical,
n is an integer ranging from 0 to 100, and
m is an integer ranging from 0 to 100, with the proviso that the sum n+m ranges from 1 to 100. In particular, the radicals R of formula (VIII) and R₁ to R₁₀ defined previously may each represent a linear or branched, saturated or unsaturated alkyl radical, especially of C₂-C₂₀, in particular C₃-C₁₆ and more particularly C₄-C₁₀, or a monocyclic or polycyclic C₆-C₁₄ and especially C₁₀-C₁₃ aryl radical, or an aralkyl radical whose aryl and alkyl residues are as defined previously. Preferably, R of formula (VIII) and R₁ to R₁₀ may each represent a methyl, ethyl, propyl, isopropyl, decyl, dodecyl or octadecyl radical, or alternatively a phenyl, tolyl, benzyl or phenethyl radical. According to one embodiment, a phenyl silicone oil of formula (VIII) with a viscosity at 25° C. of between 5 and 1500 mm²/s (i.e. 5 to 1500 cSt), and preferably with a viscosity of between 5 and 1000 mm²/s (i.e. 5 to 1000 cSt) may be used. As phenyl silicone oils of formula (VIII), it is especially possible to use phenyl trimethicones such as DC556 from Dow Corning (22.5 cSt), the oil Silbione 70663V30 from Rhône-Poulenc (28 cSt) or diphenyl dimethicones such as Belsil oils, especially Belsil PDM1000 (1000 cSt), Belsil PDM 200 (200 cSt) and Belsil PDM 20 (20 cSt) from Wacker. The values in parentheses represent the viscosities at 25° C.
the phenyl silicone oils corresponding to the following formula, and mixtures thereof:

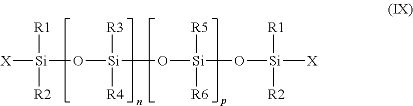

in which in formula (IX):
R₁, R₂, R₅ and R₆ are, together or separately, an alkyl radical containing 1 to 6 carbon atoms,
R₃ and R₄ are, together or separately, an alkyl radical containing from 1 to 6 carbon atoms or an aryl radical,
X is an alkyl radical containing from 1 to 6 carbon atoms, a hydroxyl radical or a vinyl radical,
n and p being chosen so as to give the oil a weight-average molecular mass of less than 200 000 g/mol, preferably less than 150 000 g/mol and more preferably less than 100 000 g/mol.

The phenyl silicones that are most particularly suitable for use in the present invention may be those corresponding to formulae (II) and especially to formulae (III), (V) and (VIII) hereinabove.

More particularly, the phenyl silicones may be chosen from phenyl trimethicones, phenyl dimethicones, phenyl-trimethylsiloxydiphenylsiloxanes, diphenyl dimethicones, diphenylmethyldiphenyltrisiloxanes and 2-phenylethyl trimethylsiloxysilicates, and mixtures thereof.

Preferably, the weight-average molecular weight of the non-volatile phenyl silicone oil used according to the present invention may range from 500 to 10 000 g/mol.

Non-Volatile Non-Silicone Oil:

As the non-volatile non-silicone oils, mention may be made of:
plant oils, for example phytostearyl esters such as phytostearyl oleate, phytostearyl isostearate and lauroyl/octyldodecyl/phytostearyl glutamate, and triglycerides formed from fatty acid esters of glycerol, in particular whose fatty acids may have chain lengths ranging from C₁₈ to C₃₆, these oils possibly being linear or branched, and saturated or unsaturated; these oils may especially be heptanoic or octanoic triglycerides, shea oil, alfalfa oil, poppy oil, pumpkin oil, millet oil, barley oil, quinoa oil, rye oil, candlenut oil, passionflower oil, shea butter oil, aloe oil, sweet almond oil, peach stone oil, groundnut oil, argan oil, avocado oil, baobab oil, borage oil, broccoli oil, calendula oil, camellina oil, carrot oil, safflower oil, hemp oil, rapeseed oil, cottonseed oil, coconut oil, marrow seed oil, wheatgerm oil, jojoba oil, lily oil, macadamia oil, corn oil, meadowfoam oil, St-John's wort oil, monoi oil, hazelnut oil, apricot kernel oil, walnut oil, olive oil, evening primrose oil, palm oil, blackcurrant pip oil, kiwi seed oil, grape seed oil, pistachio oil, pumpkin oil, quinoa oil, musk rose oil, sesame oil, soybean oil, sunflower oil, castor oil and watermelon oil, and mixtures thereof, or alternatively caprylic/capric acid triglycerides, such as those sold by the company Stéarineries Dubois or those sold under the names Miglyol 810®, 812® and 818® by the company Dynamit Nobel;
synthetic ethers containing from 10 to 40 carbon atoms;
synthetic esters, for instance the oils of formula R₁COOR₂, in which R₁ represents at least one linear or branched fatty acid residue containing from 1 to 40 carbon atoms and R₂ represents a hydrocarbon-based chain, which is especially branched, containing from 1 to 40 carbon atoms, on the condition that R₁+R₂≥10. The esters may be chosen especially from fatty acid esters of alcohols, for instance cetostearyl octanoate, isopropyl alcohol esters, such as isopropyl myristate, isopropyl palmitate, ethyl palmitate, 2-ethylhexyl palmitate, isopropyl stearate, isopropyl isostearate, isostearyl isostearate, octyl stearate, hydroxylated esters, for instance isostearyl lactate, octyl hydroxystearate, diisopropyl adipate, heptanoates, and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate, 2-ethylhexyl palmitate, alkyl benzoates, notably C₁₂-C₁₅ alkyl benzoates, polyethylene glycol diheptanoate, propylene glycol 2-diethylhexanoate, and mixtures thereof, hexyl laurate, neopentanoic acid esters, for instance isodecyl neopentanoate, isotridecyl neopentanoate, isostearyl neopentanoate, octyldodecyl neopentanoate, isononanoic acid esters, for instance isononyl isononanoate, isotridecyl isononanoate, octyl isononanoate, hydroxylated esters, for instance isostearyl lactate and diisostearyl malate;
polyol esters and pentaerythritol esters, for instance dipentaerythrityl tetrahydroxystearate/tetraisostearate;
esters of diol dimers and of diacid dimers;
copolymers of diol dimers and of diacid dimers and esters thereof, such as dilinoleyl diol dimer/dilinoleic dimer copolymers, and esters thereof;
copolymers of polyols and of diacid dimers, and esters thereof;
fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol;
C₁₂-C₂₂ higher fatty acids, such as oleic acid, linoleic acid and linolenic acid, and mixtures thereof;
dialkyl carbonates, the two alkyl chains possibly being identical or different, such as dicaprylyl carbonate; and
oils with a molar mass of between about 400 and about 10 000 g/mol, in particular about 650 to about 10 000 g/mol, in particular from about 750 to about 7500 g/mol and more particularly ranging from about 1000 to about 5000 g/mol; mention may be made especially, alone or as a mixture, of (i) lipophilic polymers such as polybutylenes, polyisobutylenes, for example hydrogenated, polydecenes and hydrogenated polydecenes, vinylpyrrolidone copolymers, such as the vinylpyrrolidone/1-hexadecene copolymer, and polyvinylpyrrolidone (PVP) copolymers, such as the copolymers of a C₂-C₃₀ alkene, such as C₃-C₂₂, and combinations thereof; (ii) linear fatty acid esters containing a total carbon number ranging from 35 to 70, for instance pentaerythrityl tetrapelargonate; (iii) hydroxylated esters such as polyglyceryl-2 triisostearate; (iv) aromatic esters such as tridecyl trimellitate; (v) esters of fatty alcohols or of branched C₂₄-C₂₈ fatty acids, such as those described in patent U.S. Pat. No. 6,491,927 and pentaerythritol esters, and especially triisoarachidyl citrate, pentaerythrityl tetraisononanoate, glyceryl triisostearate, glyceryl 2-tridecyltetradecanoate, pentaerythrityl tetraisostearate, poly(2-glyceryl) tetraisostearate or pentaerythrityl 2-tetradecyltetradecanoate; (vi) diol dimer esters and polyesters, such as esters of diol dimer and of fatty acid, and esters of diol dimer and of diacid.

In one embodiment, the non-volatile non-silicone oil may be selected from non-polar oils, preferably ether oils, and more preferably dialkyl ethers represented by the following formula

R¹—O—R²

wherein
each of R¹ and R₂ independently denotes a linear, branched or cyclic C₄₋₂₄ alkyl group, preferably C₆₋₁₈ alkyl group, and more preferably C₈₋₁₂ alkyl group. It may be preferable that R¹ and R₂ are the same.

As the linear alkyl group, mention may be made of a butyl group, a pentyl group, a hexyl group, a heptyl group, an octyl group, a nonyl group, a decyl group, an undecyl group, a dodecyl group, tridecyl group, a tetradecyl group, a pentadecyl group, a hexadecyl group, a heptadecyl group, an octadecyl group, a nonadecyl group, an eicosyl group, a behenyl group, a docosyl group, a tricosyl group, and an tetracosyl group.

As the branched alkyl group, mention may be made of a 1-methylpropyl group, 2-methylpropyl group, a t-butyl group, a 1,1-dimethylpropyl group, a 3-methylhexyl group, a 5-methylhexyl group, an 1-ethylhexyl group, an 2-ethylhexyl group, a 1-butylpentyl group, a 5-methyloctyl group, an 1-ethylhexyl group, an 2-ethylhexyl group, a 1-butylpentyl group, a 5-methyloctyl group, a 2-butyloctyl group, an isotridecyl group, a 2-pentylnonyl group, a 2-hexyldecyl group, an isostearyl group, a 2-heptylundecyl group, an 2-octyldodecyl group, a 1,3-dimethylbutyl group, a 1-(1-methylethyl)-2-methylpropyl group, a 1,1,3,3-tetramethylbutyl group, a 3,5,5-trimethylhexyl group, a 1-(2-methylpropyl)-3-methylbutyl group, a 3,7-dimethyloctyl group, and a 2-(1,3,3-trimethylbutyl)-5,7,7-trimethyloctyl group.

As the cyclic alkyl group, mention may be made of a cyclohexyl group, a 3-methylcyclohexyl group, and a 3,3,5-trimethylcyclohexyl group.

It may be preferable that the ether oil be selected from the group consisting of dicaprylyl ether, dicapryl ether, dilauryl ether, diisostearyl ether, dioctyl ether, nonyl phenyl ether, dodecyl dimethylbutyl ether, cetyl dimethylbutyl ether, cetyl isobutyl ether, and mixtures thereof.

It may be more preferable that the ether oil be selected from the group consisting of dicaprylyl ether, dicapryl ether, dilauryl ether, diisostearyl ether, dioctyl ether, and mixtures thereof.

Thus, the composition according to the present invention, in particular the oil phase thereof, may further comprise at least one non-volatile oil which is not the (a) hydrocarbon oil, preferably at least one non-volatile non-polar oil, and more preferably non-volatile ether oil.

The amount of the non-volatile oil in the composition according to the present invention may be 1% by weight or more, preferably 3% by weight or more, and more preferably 5% by weight or more, relative to the total weight of the composition.

The amount of the non-volatile oil in the composition according to the present invention may be 12% by weight or less, preferably 10% by weight or less and more preferably 8% by weight or less, relative to the total weight of the composition, with the proviso that the amount of the non-volatile oil is not zero.

The amount of the non-volatile oil in the composition according to the present invention may be from 1% to 12% by weight, preferably from 3% to 10% by weight, and more preferably from 5% to 8% by weight, relative to the total weight of the composition.

In one embodiment, the non-volatile oil in the composition according to the present invention may be selected from polar oils, for example, ester oils, such as synthetic esters as explained above. For example, the composition according to the present invention may comprise polar oil such as ester oil in an amount of 20% by weight or less, and preferably 10% by weight or less, relative to the total weight of the composition. On the other hand, the amount of polar oil such as ester oil may be 0.01% by weight or more, and preferably 0.1% by weight or more, relative to the total weight of the composition. Thus, the amount of polar oil such as ester oil may be from 0.01% to 20% by weight, and preferably from 0.1% to 10% by weight, relative to the total weight of the composition.

In another embodiment, it may be preferable that the composition according to the present invention, in particular the oil phase thereof, be free from polar oils. Thus, the composition according to the present invention may comprise no polar oil such as ester oil.

(Aromatic Ketone Compound)

The composition according to the present invention, in particular the aqueous phase thereof, may comprise at least one aromatic ketone compound. If two or more aromatic ketone compounds are used, they may be the same or different.

The aromatic ketone compound is represented by the following chemical formula (I):

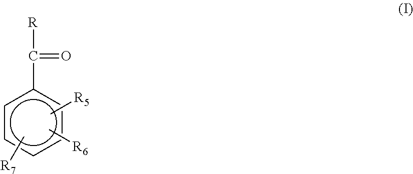

wherein
R₅, R₆ and R₇, each independently, represent a hydrogen atom, a hydroxyl group, a C₁-C₆ alkyl group, or a C₁-C₆ alkoxy group, with the proviso that at least one of R₅, R₆ and R₇ represents a hydroxyl group, and
R represents a C₁-C₆ alkyl group or an aryl group.

As the C₁-C₆ alkyl group, mention may be made of, for example, a methyl group, an ethyl group, and a propyl group. A methyl group is preferable.

As the C₁-C₆ alkoxy group, mention may be made of, for example, a methoxy group, an ethoxy group, and a propoxy group. A methoxy group is preferable.

As the aryl group, mention may be made of, for example, a phenyl group, a substituted phenyl group, a naphthyl group, and a substituted naphthyl group. As the substituent(s), mention may be made of a hydroxyl group and a C₁-C₆ alkyl group such as a methyl group. A phenyl group is preferable.

It is preferable that the R in the above chemical formula (I) represent a methyl group, an ethyl group or a phenyl group, preferably a methyl group or a phenyl group, and more preferably a methyl group.

As the aromatic ketone compound, mention may be made of, for example,
2-hydroxyacetophenone,
3-hydroxyacetophenone,
4-hydroxyacetophenone,
2,5-dihydroxyacetophenone,
2,6-dihydroxyacetophenone,
4-hydroxy-3-methoxyacetophenone,
3,4,5-trihydroxyacetophenone,
2′,2′-dihydroxybenzophenone,
2-hydroxybenzophenone,
2,2′,4,4′-tetrahydroxybenzophenone, and
3,4,2′,4′,6′-pentahydroxybenzophenone.

It is preferable that the aromatic ketone compound be hydroxyacetophenone, more preferably monohydroxyacetophenone, and even more preferably 4-hydroxyacetophenone.

The amount of the aromatic ketone compound in the composition according to the present invention may be 0.01% by weight or more, preferably 0.05% by weight or more, and more preferably 0.1% by weight or more, relative to the total weight of the composition.

The amount of the aromatic ketone compound in the composition according to the present invention may be 5% by weight or less, preferably 3% by weight or less, and more preferably 1% by weight or less, relative to the total weight of the composition, with the proviso that the amount of the aromatic ketone compound is not zero.

The amount of the aromatic ketone compound in the composition according to the present invention may be from 0.01% to 5% by weight, preferably from 0.05% to 3% by weight, and more preferably from 0.1% to 1% by weight, relative to the total weight of the composition.

(Polyglyceryl Fatty Acid Ester)

The composition according to the present invention, in particular the aqueous phase thereof, may comprise at least one polyglyceryl fatty acid ester. If two or more polyglyceryl fatty acid esters are used, they may be the same or different.

The polyglyceryl fatty acid ester may be chosen from the mono, di and tri esters of a linear or branched, saturated or unsaturated fatty acid, preferably saturated fatty acid, including from 2 to 28 carbon atoms, preferably from 4 to 20 carbon atoms, and more preferably from 6 to 12 carbon atoms, such as lauric acid, oleic acid, stearic acid, isostearic acid, capric acid, caprylic acid, and myristic acid.

It is preferable that the polyglyceryl fatty acid ester have a polyglycerol moiety derived from 2 to 6 glycerols, more preferably from 4 to 6 glycerols, and even more preferably 5 or 6 glycerols. In other words, it is preferable that the polyglyceryl fatty acid ester comprise from 2 to 6 polyglyceryl units, more preferably 4 to 6 polyglyceryl units, and even more preferably 5 or 6 polyglyceryl units.

In the composition according to the present invention, the polyglyceryl fatty acid ester may be present in the aqueous phase. Therefore, the polyglyceryl fatty acid ester may be relatively hydrophilic.

Thus, the polyglyceryl fatty acid ester may have an HLB (Hydrophilic Lipophilic Balance) value of 8.0 or more, preferably 9.0 or more, and more preferably 10.0 or more. If two or more polyglyceryl fatty acid esters are used, the HLB value is determined by the weighted average of the HLB values of all the polyglyceryl fatty acid esters.

The term HLB (“hydrophilic-lipophilic balance”) is well known to those skilled in the art, and reflects the ratio between the hydrophilic part and the lipophilic part in the molecule. HLB values can be calculated with the formula HLB=20*(1−S/A), where S is the saponification number of the ester and A is the neutralization number of the fatty acid.

The polyglyceryl fatty acid ester may be selected from polyglyceryl fatty acid monoesters.

The polyglyceryl fatty acid monoester may have an HLB value of from 8.0 to 17.0, preferably from 9.0 to 16.0, and more preferably from 10.0 to 15.0. If two or more polyglyceryl fatty acid monoesters are used, the HLB value is determined by the weighted average of the HLB values of all the polyglyceryl fatty acid monoesters.

The polyglyceryl fatty acid monoester may be selected from the group consisting of PG4 oleate (HLB: 8.8), PG4 laurate (HLB: 10.3), PG4 isostearate (HLB: 8.2), PG5 laurate (HLB: 10.5), PG6 isostearate (HLB: 10.8), PG4 caprylate (HLB: 14), PG4 caprate (HLB: about 15), PG5 myristate (HLB: 15.4), PG5 stearate (HLB: 15), PG5 oleate (HLB: 12.2), PG6 caprylate (HLB: 14.6), PG6 caprate (HLB: 13.1), PG6 laurate (HLB: 14.1) and mixtures thereof.

It is preferable for the polyglyceryl fatty acid ester be selected from polyglyceryl fatty acid diesters.

The polyglyceryl fatty acid diester may have an HLB value of from 8.0 to 13.0, preferably from 9.0 to 12.0, and more preferably from 10.0 to 11.0. If two or more polyglyceryl fatty acid diesters are used, the HLB value is determined by the weighted average of the HLB values of all the polyglyceryl fatty acid diesters.

The polyglyceryl fatty acid diester may be selected from the group consisting of PG-6 distearate (HLB: 8), PG-6 dioleate (HLB: 9.8), PG-6 dicaprate (HLB: 10.2), and mixtures thereof.

As PG6 dicaprate, for example, Sunsoft Q-102H-C marketed by Taiyo Kagaku Co., Ltd. may be used. As PG6 dioleate, Sunsoft Q-172H-C marketed by Taiyo Kagaku Co. Ltd. may be used. As PG6 distearate, Emalex DSG-6 marketed by Nihon Emulsion Co., Ltd. may be used.

The amount of the polyglyceryl fatty acid ester in the composition according to the present invention may be 0.001% by weight or more, preferably 0.005% by weight or more, and more preferably 0.01% by weight or more, relative to the total weight of the composition.

The amount of the polyglyceryl fatty acid ester in the composition according to the present invention may be 1% by weight or less, preferably 0.5% by weight or less and more preferably 0.1% by weight or less, relative to the total weight of the composition, with the proviso that the amount of the polyglyceryl fatty acid ester is not zero.

The amount of the polyglyceryl fatty acid ester in the composition according to the present invention may be from 0.001% to 1% by weight, preferably from 0.005% to 0.5% by weight, and more preferably from 0.01% to 0.1% by weight, relative to the total weight of the composition.

(Surfactant)

The composition according to the present invention may comprise at least one additional surfactant which is different from the polyglyceryl fatty acid ester. If two or more additional surfactants are used, they may be the same or different.

However, it may be preferable that the amount of the additional surfactant is small.

The amount of the additional surfactant may be 1% by weight or less, preferably 0.5% by weight or less, and more preferably 0.3% by weight or less, relative to the total weight of the composition according to the present invention. It is in particular preferable that the composition according to the present invention comprise no additional surfactant.

(Other Ingredients)

The composition according to the present invention may also include at least one optional or additional ingredient.

The amount of the optional or additional ingredient(s) is not limited, but may be from 0.01% to 30% by weight, preferably from 0.1% to 20% by weight, and more preferably from 1% to 10% by weight, relative to the total weight of the composition according to the present invention.

The optional or additional ingredient(s) may be selected from the group consisting of anionic, cationic, nonionic, or amphoteric polymers; organic or inorganic UV filters; peptides and derivatives thereof; protein hydrolyzates; swelling agents; penetrating agents; thickeners; suspending agents; sequestering agents; opacifying agents; dyes; vitamins or provitamins; fragrances; preservatives, co-preservatives, stabilizers; and mixtures thereof.

The composition according to the present invention may include one or several cosmetically acceptable organic solvents, which may be alcohols: in particular monovalent alcohols such as ethyl alcohol, isopropyl alcohol, benzyl alcohol, and phenylethyl alcohol; diols such as ethylene glycol, propylene glycol, and butylene glycol; other polyols such as glycerol, sugar, and sugar alcohols; and ethers such as ethylene glycol monomethyl, monoethyl, and monobutyl ethers, propylene glycol monomethyl, monoethyl, and monobutyl ethers, and butylene glycol monomethyl, monoethyl, and monobutyl ethers.

The organic solvent(s) may then be present in a concentration of from 0.01% to 20% by weight, preferably from 0.1% to 15% by weight, and more preferably from 1% to 10% by weight, relative to the total weight of the composition.

The pH of the composition according to the present invention may be controlled. The pH may be, for example, from 3 to 11, preferably from 3.5 to 9, and more preferably from 4 to 7.

The pH may be adjusted to the desired value using at least one acidifying agent and/or at least one basifying agent.

The acidifying agents can be, for example, mineral or organic acids, for instance hydrochloric acid, orthophosphoric acid, carboxylic acid, for instance tartaric acid, citric acid, lactic acid, or sulphonic acids.

The basifying agent can be, for example, ammonium hydroxide, alkali metal hydroxide, alkali earth metal hydroxide, alkali metal carbonates, alkanolamines such as mono-, di-, and triethanolamines, and also their derivatives, preferably sodium or potassium hydroxide and compounds of the formula below:

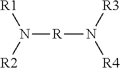

wherein
R denotes an alkylene such as propylene optionally substituted by a hydroxyl or a C₁-C₄ alkyl radical, and R₁, R₂, R₃, and R₄ independently denote a hydrogen atom, an alkyl radical, or a C₁-C₄ hydroxyalkyl radical, which may be exemplified by 1,3-propanediamine and derivatives thereof. Arginine, urea, and monoethanolamine may be preferable.

The acidifying or basifying agent may be present in an amount ranging from less than 5% by weight, preferably from 1% by weight or less, and more preferably from 0.1% by weight or less, relative to the total weight of the composition.

(Form)

The composition according to the present invention can have two visually distinct phases. One of the two phases is an oil phase, and the other is an aqueous phase.

The amount of the oil phase in the composition according to the present invention may be 7% by weight or more, and preferably 15% by weight or more, relative to the total weight of the composition.

The amount of the oil phase in the composition according to the present invention may be 70% by weight or less, and preferably 65% by weight or less, relative to the total weight of the composition, with the proviso that the amount of the oil phase is not zero.

The amount of the oil phase in the composition according to the present invention may be from 7% to 70% by weight, and preferably from 15% to 65% by weight, relative to the total weight of the composition.

The oil phase in the composition according to the present invention may further comprise any hydrophobic ingredient in addition to the (a) hydrocarbon oil as well as the optional non-volatile oil.

For example, the oil phase of the composition according to the present invention may comprise at least one lipophilic or liposoluble cosmetic active ingredient. A single type of such cosmetic active ingredient, or two or more different types of such cosmetic active ingredient may be used in combination.

The amount of the aqueous phase in the composition according to the present invention may be 30% by weight or more, and preferably 35% by weight or more, relative to the total weight of the composition.

The amount of the aqueous phase in the composition according to the present invention may be 93% by weight or less, and preferably 85% by weight or less, relative to the total weight of the composition, with the proviso that the amount of the aqueous phase is not zero.

The amount of the aqueous phase in the composition according to the present invention may be from 30% to 93% by weight, and preferably from 35% to 85% by weight, relative to the total weight of the composition.

The aqueous phase in the composition according to the present invention may further comprise any hydrophilic ingredient in addition to the (b) ester of citric acid and (c) water.

For example, the aqueous phase of the composition according to the present invention may comprise at least one hydrophilic or water-soluble cosmetic active ingredient. A single type of such cosmetic active ingredient, or two or more different types of such cosmetic active ingredient may be used in combination.

In addition, the aqueous phase may comprise at least one pH adjuster such as an acid and a base, and/or at least one organic solvent such as diol.

The composition according to the present invention can be transformed into a single phase composition when being mixed. The composition according to the present invention after mixing can be of the O/W type or W/O type, preferably in the form of an O/W or W/O liquid composition, and more preferably in the form of an O/W or W/O emulsion.

The weight ratio of the oil phase/the aqueous phase may be from 50/50 to 5/95, preferably from 50/50 to 10/90, and more preferably from 50/50 to 15/85.

If the specific gravity of the oil phase is smaller than that of the aqueous phase, the oil phase is present on the aqueous phase, in the composition according to the present invention.

Typically, the oil phase is present on the aqueous phase, at rest, because the specific gravity of oil tends to be smaller than that of water.

[Preparation]

The composition according to the present invention can be prepared by forming an oil phase by providing (a) hydrocarbon oil or mixing the ingredients, which include (a) hydrocarbon oil(s), for the oil phase, forming an aqueous phase by mixing the ingredients, which include (b) ester of citric acid, and (c) water, for the aqueous phase, and combining the oil phase and the aqueous phase.

For example, the composition according to the present invention can be prepared by the process comprising:
(i) providing or mixing
   (a) at least one hydrocarbon oil
   with at least one optional ingredient to form an oil phase;
(ii) mixing
   (b) at least one ester of citric acid; and
   (c) water
   with at least one optional ingredient to form an aqueous phase; and
(iii) combining the oil phase and the aqueous phase.

The mixing step can be performed by any conventional means.

It is preferable that the step of combining the oil phase and the aqueous phase be performed gently such that the oil phase and the aqueous phase are slowly poured into a vessel such as a container.

[Use and Process]

When using the composition according to the present invention, it will be mixed by, for example, shaking with the hands (hand-shaking). After mixing the composition according to the present invention, the composition can form a single phase composition. The single phase composition can be applied onto a keratin substance to perform target cosmetic effects.

The keratin substance may be skin, nails, mucosae such as lips, or keratin fibers such as eyebrow and eyelashes.

The composition according to the present invention may preferably be used as a cosmetic composition, more preferably a cleansing cosmetic composition, and even more preferably a cleansing cosmetic composition for skin and eyelashes.

The composition according to the present invention can provide cosmetic effects such as make-up removal.

The composition according to the present invention can also provide a good texture such as a smooth feeling to touch, and therefore, it can provide a smooth skin finish.

The composition according to the present invention is comfortable because it is less irritating, and it is mild on the skin, in particular sensitive skin such as the skin around the eyes.

The skin here encompasses facial skin, neck skin, and the scalp. The composition according to the present invention may also be used for mucosae such as lips.

The composition according to the present invention can be used as it is (as a topical product), or can be used by being impregnated into a porous substrate such as a non-woven fabric preferably made from cellulose fibers such as cotton.

In particular, the composition according to the present invention may be intended for application onto a keratin substance such as skin, lips, eyebrows and eyelashes. Thus, the composition according to the present invention can be used for a cosmetic process for a keratin substance such as skin, lips, eyebrows and eyelashes, preferably skin and eyelashes.

The composition according to the present invention may be used for caring or cleansing, not for making up, a keratin substance. It is preferable that the composition according to the present invention be used for skin care products such as a lotion or cleansing products such as a make-up remover, not for make-up products such as a foundation. Preferably the composition according to the present invention does not comprise iron oxide, or comprises iron oxide in an amount of 0.5% by weight or less, more preferably 0.2% by weight or less, even more preferably 0.1% by weight or less, relative to the total weight of the composition according to the present invention.

The cosmetic process for a keratin substance according to the present invention may comprise, at least, the steps of mixing the composition according to the present invention to form a single phase composition; and applying the single phase composition onto the keratin substance.

The single phase composition does not have two visually distinct phases. Instead, the single phase composition has a single phase which is typically an emulsion phase. The emulsion phase may not be transparent, but it may be translucent or opaque.

The present invention may relate to the use of:
(a) at least one hydrocarbon oil, and
(b) at least one ester of citric acid;
in a two-phase composition comprising an aqueous phase comprising the (b) ester(s) of citric acid and (c) water and an oil phase comprising the (a) hydrocarbon oil(s), wherein the amount of the (a) hydrocarbon oil(s) in the composition is 6% by weight or more, preferably 7% by weight or more, and more preferably 8% by weight or more, relative to the total weight of the composition, in order to enhance make-up removability and/or smoothness of the composition.

### EXAMPLES

The present invention will be described in a more detailed manner by way of examples. However, these examples should not be construed as limiting the scope of the present invention. The examples below are presented as non-limiting illustrations in the field of the present invention.

### Examples 1-2 and Comparative Examples 1-3

The following compositions according to Examples 1-2 and Comparative Examples 1-3 shown in Table 1 were prepared by mixing the ingredients shown in Table 1 as follows.
(1) mixing the ingredients of Oil Phase (OP in Table 1) at about 25° C. to form a uniform mixture of Oil Phase ingredients;
(2) mixing the ingredients of Aqueous Phase (AP in Table 1) at about 60° C. to form a uniform mixture of Aqueous Phase ingredients; and
(3) pouring the above into a package in order of the Aqueous Phase ingredients, then the Oil Phase ingredients.

It should be noted that “hydroxyacetophenone” means 4-hydroxyacetophenone.

The numerical values for the amounts of the ingredients shown in Table 1 are all based on “% by weight” as active raw materials.

TABLE 1

| | | | | Comp. | Comp. | Comp |
|---|---|---|---|---|---|---|
| | | Ex. 1 | Ex. 2 | Ex. 1 | Ex. 2 | Ex. 3 |
| OP | Isohexadecane | 9 | 27.5 | 5 | — | 9 |
| | Dicaprylyl | 6 | 10 | — | — | 6 |
| | Ether | | | | | |
| | Isopropyl | — | — | 10 | 15 | — |
| | Palmitate | | | | | |
| | Isododecane | — | 11.5 | — | — | — |
| | Triethyl Citrate | — | 1 | — | — | — |
| | Total | 15 | 50 | 15 | 15 | 15 |
| AP | Hydroxy- | 0.425 | 0.25 | 0.425 | 0.425 | 0.425 |
| | acetophenone | | | | | |
| | Butylene Glycol | 1.7 | 1 | 1.7 | 1.7 | 1.7 |
| | Citric Acid | 0.034 | 0.02 | 0.034 | 0.034 | 0.034 |
| | Sodium Citrate | 0.0935 | 0.055 | 0.0935 | 0.0935 | 0.0935 |
| | Sodium Chloride | 0.425 | 0.25 | 0.425 | 0.425 | 0.425 |
| | Polyglyceryl-6 | 0.0425 | 0.005 | 0.0425 | 0.0425 | 0.0425 |
| | Dicaprate | | | | | |
| | Myrtrimonium | 0.0595 | 0.035 | 0.0595 | 0.0595 | 0.0595 |
| | Bromide | | | | | |
| | Triethyl Citrate | 1.7 | 1 | 1.7 | 1.7 | — |
| | Water | qsp 85 | qsq 50 | qsp 85 | qsp 85 | qsp 85 |
| | Total | 85 | 50 | 85 | 85 | 85 |

| | | | | | |
|---|---|---|---|---|---|
| OP/AP Weight Ratio | 15/85 | 50/50 | 15/85 | 15/85 | 15/85 |
| Smoothness | 4.2 | 4.2 | 1.3 | 2.1 | 3.6 |
| Make-up Removability | 3.7 | 4.2 | 1 | 0.7 | 1.8 |

OP: Oil Phase
AP: Aqueous Phase

[Evaluation]

The compositions according to Examples 1-2 and Comparative Examples 1-3 were evaluated as follows.

(Smoothness)

Each of the compositions according to Examples 1-2 and Comparative Examples 1-3 was shaken to form an emulsion. 1 mL of the emulsion was applied onto a cotton sheet, and the forearm of three panelists was wiped with the cotton sheet ten times. The panelists evaluated the smoothness on the skin after wiping the forearm with a grade of from 1 (very unsmooth=sticky) to 5 (very smooth), and the scores of the grades were averaged.

The results are shown in Table 1.

(Make-Up Removability)

A mascara product (MAYBELLINE Volum'Express Hypercurl Waterproof N 01 Black) was applied onto the forearm of three panelists. The mascara product was dried for 30 minutes to form a mascara film on the forearm.

Each of the compositions according to Examples 1-2 and Comparative Examples 1-3 was shaken to form an emulsion. 1 mL of the emulsion was applied onto a cotton sheet, and the cotton sheet was placed on the mascara film on the forearm for 10 second. Then, the mascara film was wiped once. The panelists evaluated the make-up removability with a grade of from 1 (very poor) to 5 (excellent), and the scores the grades were averaged.

The results are shown in Table 1.

(Summary)

Example 1 shows that the use of a combination of triethyl citrate and isohexadecane in an amount of 6% by weight or more relative to the total weight of the composition according to Example 1 can provide good make-up removal efficacy without discomfort such as stickiness.

The two-phase composition according to Example 1 was able to be transformed into a single phase composition, when being used, and was able to return to the two-phase composition, when not being used.

Example 2 shows that the use of a combination of triethyl citrate and isohexadecane in a more amount can also provide good make-up removal efficacy without discomfort such as stickiness. The two-phase composition according to Example 2 was able to be transformed into a single phase composition, when being used, and was able to return to the two-phase composition, when not being used.

Comparative Example 1 shows that when the amount of isohexadecane is less than 6% by weight relative to the total weight of the composition according to Comparative Example 1, the make-up removal efficacy is deteriorated, and stickiness was caused.

Comparative Example 2 shows that when isohexadecane is not used, the make-up removal efficacy is more deteriorated, and stickiness was caused.

Comparative Example 3 shows that when triethyl citrate is not used, the make-up removal efficacy is more deteriorated, and stickiness was caused.

1. A two phase composition comprising:
an oil phase comprising (a) at least one hydrocarbon oil; and
an aqueous phase comprising (b) at least one ester of citric acid, and (c) water,
wherein
the amount of the (a) hydrocarbon oil in the composition is 6% by weight or more, relative to the total weight of the composition.

2. The composition according to claim 1, wherein the composition is capable of transforming into a single phase composition.

3. The composition according to claim 2, wherein the single phase composition is capable of transforming into a two phase composition.

4. The composition according to claim 1, wherein the (a) hydrocarbon oil is a volatile hydrocarbon oil.

5. The composition according to claim 1, wherein the amount of the (a) hydrocarbon oil(s) in the composition is from 6% to 50% by weight, relative to the total weight of the composition.

6. The composition according to claim 1, wherein the (b) ester of citric acid is represented by the following chemical formula (I): 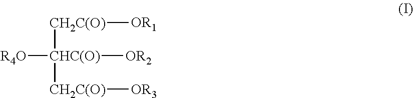
wherein
R₁, R₂ and R₃ independently represent a hydrogen atom, a saturated or unsaturated, linear or branched C₁-C₃₀ hydrocarbon group, or a saturated or unsaturated cyclic C₃-C₃₀ hydrocarbon group, wherein at least one of R₁, R₂ and R₃ is not a hydrogen atom, and
R₄ represents a hydrogen atom or an R′₄—CO— group in which R′4 represents a saturated or unsaturated, linear or branched C₁-C₈ hydrocarbon group, or a saturated or unsaturated cyclic C₃-C₈ hydrocarbon group.

7. The composition according to claim 1, wherein the (b) ester of citric acid is selected from the group consisting of triethyl citrate, tributyl citrate, trioctyl citrate, triethyl acetylcitrate, tribuyl acetylcitrate, tri(2-ethylhexyl) acetylcitrate, and a mixture thereof.

8. The composition according to claim 1, wherein the amount of the (b) ester (s) of citric acid in the composition is from 0.1% to 10% by weight, relative to the total weight of the composition.

9. The composition according to claim 1, wherein the amount of the (c) water in the composition is from 30% to 93% by weight, relative to the total weight of the composition.

10. The composition according to claim 1, wherein the composition further comprises at least one non-volatile oil which is not the (a) hydrocarbon oil.

11. The composition according to claim 1, wherein the composition further comprises polar oil in an amount of from 0.01% to 20% by weight, relative to the total weight of the composition.

12. The composition according to claim 1, wherein the amount of the oil phase in the composition is from 7% to 70% by weight, relative to the total weight of the composition.

13. The composition according to claim 1, wherein the amount of the aqueous phase in the composition is from 30% to 93% by weight, relative to the total weight of the composition.

14. The composition according to claim 1, wherein the composition is a cosmetic composition.

15. A cosmetic process for a keratin substance, comprising:
mixing the composition according to claim 1 to form a single phase composition; and
applying the single phase composition onto the keratin substance.

16. The composition according to claim 1, wherein the aqueous phase further comprises at least one aromatic ketone compound.

17. The composition according to claim 1, wherein the aqueous phase further comprises at least one polyglyceryl fatty acid ester.
